(19) United States
(12) Patent Application Publication
(10) Pub. No.: US 2018/0280205 A1
(43) Pub. Date: Oct. 4, 2018

(54) ULTRASONIC BONDING OF ELASTIC FIBER

(71) Applicant: INVISTA NORTH AMERICA S.A R.L., WILMINGTON, DE (US)

(72) Inventor: Ronald D. BING-WO, Waynesboro, VA (US)

(73) Assignee: INVISTA NORTH AMERICA S.A R.L., WILMINGTON, DE (US)

(21) Appl. No.: 15/540,397

(22) PCT Filed: Dec. 29, 2015
(86) PCT No.: PCT/US15/67830

§ 371 Date: Jun. 28, 2017

Related U.S. Application Data
(60) Provisional application No. 62/097,402, filed on Dec. 29, 2014.

(51) Int. Cl. A61F 13/15; A61F 13/49; B29C 65/08; B29C 65/00

(52) U.S. Cl. CPC A61F 13/15699; A61F 13/4902; B29C 65/086; B29L 2031/4878; B29C 66/81433; B29C 66/83413; A61F 2013/15869; B29C 66/344

(57) ABSTRACT

Elastic band products of high durability and quality are disclosed. Also disclosed are methods of producing durable, high quality elastic band products using modified anvil blocks, including shaped anvil blocks, anvil blocks with lateral protuberances and/or anvil blocks with teeth. The disclosed methods result in reduced elastic yarn thread slippage both during production and in the finished elastic band product.

### CROSS-REFERENCE TO RELATED APPLICATION

This application claims the benefit of U.S. Provisional application Ser. No. 62/097,402, filed Dec. 29, 2014, the disclosure of which is specifically incorporated herein by reference in their entity.

### FIELD OF THE INVENTION

The present invention relates generally to elastic bands for use with textiles. More particularly, the present invention is directed to methods of more efficiently producing elastic band products with enhanced durability.

### BACKGROUND OF THE INVENTION

Elastic bands, such as elastic waist bands and elastic leg bands, are well-known for their use in textile articles. For example, trousers and other pants may incorporate elastic waist bands in an effort to keep the clothing close to the skin while allowing for potential expansion. Specifically, trousers and other pants with elastic waist bands allow a user to comfortably wear the trousers even while the user's waistline may grow (or shrink) over time. Such clothing can be especially helpful for quickly growing young children, allowing the child to wear the trousers for a longer period of time than if the trousers did not include an elastic waist band.

Elastic bands are especially useful in articles such as disposable diapers (both child and adult). An important requirement of disposable diapers is that they contain any fecal matter or urine contained therein without releasing such matter or urine. In order to accomplish this task, disposable diapers generally incorporate elastic waist and leg bands in an effort to keep the diaper close to the skin while at the same time maintaining comfort and safely keeping contents within the diaper.

There are many methods of making the various types of elastic bands used in, e.g., disposable diapers. For example, some diapers use chemical bonding or adhesive agents, such as glue, to maintain the various elements of the elastic band product in the proper position(s). This method may be undesirable because the chemical bonding or adhesive agents may be messy and impede production. The chemical bonding or adhesive agents may also be unsightly on the finished product. Moreover, chemical bonding or adhesive agents increase the cost of the elastic band products. Additionally, the glue dispensing part of the production machine may break, thereby causing a disruption in the production line.

A different method of making elastic band products is disclosed in U.S. Pat. No. 6,291,039 to Combe et al., which teaches a ruffling slide and method for making that seeks “to provide, by prior ruffling, a local functional zone of limited possible elastic stretching.” Combe's method requires a duo of wheels, an “anvil” (or “base”) wheel and a “forming wheel.” An elastic yarn thread is stretched out length-wise and sandwiched between two textile sheets and is then fed through a bonding machine, i.e., the combination of the anvil and forming wheels. The elastic yarn thread is guided and kept in position through use of anvil blocks located on the anvil wheel. Specifically, the elastic yarn thread fits between a set of anvil blocks which maintains the location of the elastic yarn thread between the two textile sheets. Combe's anvil blocks may also minimally assist in maintaining the elastic yarn thread in a stretched state during production of the elastic band product. Moreover, when Combe's anvil blocks contact the forming wheel, the two textile sheets are pressed together and then bonded through heat sealing (either direct heat application or caused by ultrasound), thereby creating connecting portions. The apparent purpose of the connecting portions is to maintain the location of the elastic yarn thread in the finished elastic band product so that the elastic yarn thread does not slip though the sandwiched textile sheets when the elastic band product is stretched during use. A plurality of anvil blocks may be located on the anvil wheel, thereby allowing for production of an elastic band product containing multiple elastic yarn threads.

However, even in systems such as the Combe system, the elastic yarn thread is susceptible to slipping (also known as “creeping”) out of place either during production or use of the elastic band product. If slippage occurs during production, the production line must be stopped and reset, thereby increasing production costs; if slippage occurs during use, the elastic band product loses its elastic properties, thereby resulting in the failure of a disposal diaper. It is obviously highly desirable by the user of the product to avoid any failure, even a minor failure, of a disposable diaper.

### SUMMARY OF THE INVENTION

Included are articles and methods for producing elastic band products comprising one or more elastic yarn threads sandwiched between two textile sheets through use of a bonder apparatus, such as a rotary ultrasonic bonder, comprising a horn wheel and an anvil wheel; the horn wheel may be an ultrasonic horn wheel, and the anvil wheel includes anvil blocks which may be textured with teeth or contain lateral protuberances. Moreover, these objects and others not particularly set forth above, are achieved by producing elastic band products using anvil wheels with grooves or convex ridges between the anvil blocks. Additionally, these objects and others not particularly set forth above, are achieved by staggering the pattern of anvil blocks on the anvil wheel. Further, these objects and others not particularly set forth above, are achieved by altering the shape of the anvil blocks. Even further, these objects and others not particularly set forth above, are achieved by including convex members that run around the circumference of the horn wheel in specific locations so as to line up between the anvil blocks on the anvil wheel.

Desirably, the anvil blocks will be staggered across the anvil wheel, with each anvil block such as being textured with teeth, altered to include lateral protuberances and/or altered in shape (collectively, “modified anvil blocks”). A groove, such as angular/profiled, will run around the circumference of the anvil wheel, between the anvil blocks; the groove may be present around the entire anvil wheel, or the groove may be present only on some portions of the anvil wheel, e.g., between the anvil blocks, or the groove may be present between only some anvil blocks. Convex members may run around the circumference of the horn wheel, with each convex member positioned so as to lie in the spaces between the anvil blocks. The convex members may be present around the entire horn wheel, or the convex members may be present only on some portions of the horn wheel, e.g., positioned so as to line up only between the anvil blocks on the anvil wheel, or the convex members may be positioned to line up only between some anvil blocks on the anvil wheel. The horn wheel may provide ultrasonic waves to cause the two textile sheets to bond together, around the elastic yarn thread(s).

### BRIEF DESCRIPTION OF THE FIGURES

FIG. 1A is a perspective view of an embodiment of the bonding machine comprising an anvil wheel and horn wheel according to the present invention.

FIG. 1B is a sectional elevated view of an embodiment of the bonding machine comprising an anvil wheel and a horn wheel according to the present invention, wherein the horn wheel comprises convex members. FIG. 1B illustrates the interaction between the convex members of the horn wheel, the elements of the anvil wheel and the elastic yarn thread.

FIG. 1C is an elevated side view of an embodiment of the bonding machine comprising an anvil wheel and a horn wheel according to the present invention, wherein the horn wheel comprises convex members. FIG. 1C illustrates the interaction between the convex members of the horn wheel, the elements of the anvil wheel and the elastic yarn thread.

FIG. 2 is a perspective view of a basic embodiment of the anvil blocks according to the present invention.

FIG. 3A is a sectional elevated view of an embodiment of the anvil blocks according to the present invention, taken along Line C-C of FIG. 2.

FIG. 3B is a sectional elevated view of an embodiment of the anvil blocks according to the present invention, taken along Line C-C of FIG. 2.

FIG. 3C is a sectional elevated view of an embodiment of the anvil blocks according to the present invention, taken along Line C-C of FIG. 2.

FIG. 4A is a perspective view of an embodiment of the modified anvil blocks according to the present invention.

FIG. 4B is a perspective view of an embodiment of the modified anvil blocks according to the present invention.

FIG. 5A is a sectional elevated view of an embodiment of the anvil blocks according to the present invention, taken along Line C-C of FIG. 2.

FIG. 5B is a sectional elevated view of an embodiment of the anvil blocks according to the present invention, taken along Line C-C of FIG. 2.

FIG. 5C is a sectional elevated view of an embodiment of the anvil blocks according to the present invention, taken along Line C-C of FIG. 2.

FIG. 5D is a sectional elevated side view of an embodiment of the anvil blocks according to the present invention, taken along Line D-D of FIG. 2.

FIG. 5E is a sectional elevated side view of an embodiment of the anvil blocks according to the present invention, taken along Line D-D of FIG. 2.

FIG. 5F is a sectional elevated side view of an embodiment of the anvil blocks according to the present invention, taken along Line D-D of FIG. 2. FIG. 5F illustrates the interaction between the convex tapered step ridge of the anvil wheel, the elements of the horn wheel and the elastic yarn thread.

FIG. 6 is an elevated side view of the bonding machine, textile sheets and elastic yarn thread according to the present invention.

FIG. 7 is a sectional top plan view of the elastic band product according to the present invention.

### DETAILED DESCRIPTION OF THE INVENTION

The following description of certain embodiments is presented to describe the present invention and is not to be construed as limiting the scope of the appended claims in any manner whatsoever.

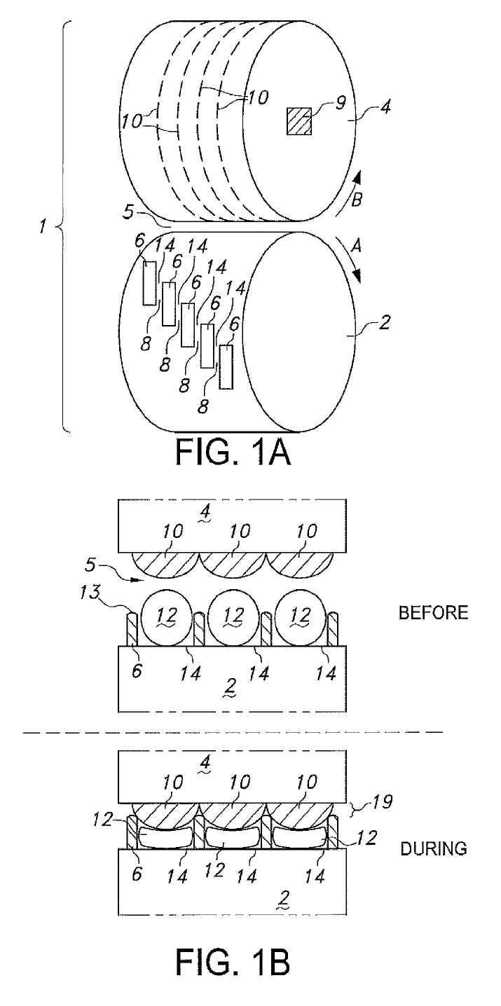

As best shown in FIG. 1A, a bonding machine 1 comprises an anvil wheel 2 and a horn wheel 4, spaced so as to form an entry path 5 therebetween; the wheels may rotate in opposite directions, e.g., the anvil wheel 2 may rotate in a clockwise manner A, and the horn wheel 4 may rotate in a counterclockwise manner B. Such opposite rotations allow material to be fed through and between the wheels 2, 4 via the entry path 5. The bonding machine 1 may be a rotary ultrasonic bonder, such as a rotary ultrasonic cantilevered bonder. However, any bonding machine known in the art may be used, e.g., a non-contact bonder or a contact bonder

The anvil wheel 2 includes a set of two or more anvil blocks 6. The anvil blocks 6 may lay across the surface of the anvil wheel 2 in an offset or staggered manner, as shown, so as to reduce vibrational shock generated when the anvil blocks 6 either make contact or are in very close proximity with the horn wheel 4 or its elements. However, it is envisioned that the anvil blocks 6 may also lay across the surface of the anvil wheel 2 so as to make a straight line across the anvil wheel 2. Moreover, the anvil blocks 6 may lay across the surface of the anvil wheel 2 in any useful pattern. Additional sets of two or more anvil blocks 6 may be located at regular intervals (not shown) across the surface of the anvil wheel 2. For example, if an anvil wheel 2 contains two sets of anvil blocks 6, the sets may be 180° apart from each other; if an anvil wheel 2 contains three sets of anvil blocks 6, the sets may be 120° apart from each other. It is also envisioned that the sets of anvil blocks 6 may be located at irregular intervals from each other. For example, an anvil wheel 2 containing three sets of anvil blocks 6 may have spacings between the first and second sets of 1000, spacings between the second and third sets of 100° and spacings between the third and first sets of 1600.

In an embodiment, the anvil wheel 2 contains at least one set of anvil blocks 6, having at least two anvil blocks 6. In an embodiment, the anvil wheel 2 contains at least thirty-five sets of anvil blocks 6, each set having at least forty anvil blocks 6. It is also envisioned that the number of anvil blocks 6 per set varies between the sets.

Yarn spaces 8 are the three dimensional empty gaps formed between the anvil blocks 6 and the yarn path 14, the yarn path 14 being a structure defined as that portion of the surface of the anvil wheel 4 that runs between two adjacent anvil blocks 6. The anvil blocks 6 are spaced apart from each other based on the thickness of the elastic yarn thread used in that particular application, and the anvil blocks 6 may be modular across the surface of the anvil wheel 2 or fixed in place across the surface of the anvil wheel 2. The width of the yarn space 8 is dictated by the distance by which the anvil blocks 6 are spaced apart from each other. The yarn space 8 may be at least slightly narrower than the diameter of the elastic yarn thread being used (as measured from when the elastic yarn thread is in a stretched state) so that the elastic yarn thread fits snugly into the yarn space 8 and contacts the anvil blocks 6 and the yarn path 14. Examples of a suitable space between the anvil blocks 6 include between about 0.01 mm and about 0.50 mm, between about 0.05 mm and about 0.40 mm, between about 0.10 mm and about 0.30 mm, and between about 0.15 mm and about 0.25 mm. In an embodiments, the space between the anvil blocks 6 is about 0.17 mm or about 0.24 mm, depending on the thickness of the elastic yarn thread and the amount of stretch placed on the elastic yarn thread. In some embodiments, the space between different anvil blocks 6 may vary. The number of yarn spaces 8 in a set of anvil blocks 6 is one less than the number of anvil blocks 6 in that set.

The horn wheel 4 may includes a means 9 for bonding textile sheets together. The means 9 may be a means for generating ultrasonic waves but may also include heating elements (not shown).

As best shown in FIGS. 1A-1C, the horn wheel 4 also may include convex members 10 on the circumference of the horn wheel 4. As best shown in FIG. 1B, the horn wheel 4 is aligned with the anvil wheel 2 so that the convex members 10 on the horn wheel 4 line up between the anvil blocks 6 and therefore line up with the yarn paths 14 of the anvil wheel 2. The “Before” sections of FIGS. 1B and 1C show the wheels 2, 4 prior to rotating (Y and Z on FIG. 1C) the convex members 10 into position between the anvil blocks 6 and over the yarn paths 14. As shown in the “During” section of FIGS. 1B and 1C, as the wheels 2, 4 rotate (Y and Z in FIG. 1C), the anvil blocks 6 come into contact with the hem wheel 4 at the anvil/horn interface 19. At the same time, convex members 10 serve to press the elastic yarn thread 12 against the yarn paths 14, thereby reducing slips. In FIG. 1C, arrow X shows the direction of movement of the elastic yarn thread 12 through the entry path 5 as the wheels 2, 4 rotate. In certain embodiments, the height of the convex members 10, as measured from the surface of the horn wheel 4, may be between about 0.01 mm and about 0.50 mm, between about 0.05 mm and about 0.40 mm, between about 0.10 mm and about 0.30 mm, and between about 0.15 mm and about 0.25 mm. In some certain embodiments, the convex member 10 has a height of between about 0.20 mm and about 0.25 mm, such as about 0.24 mm in height. It should be understood that the height of the convex member 10 is also dependent on the thickness of the elastic yarn thread, the amount of stretch placed on the elastic yarn thread and the height of the anvil blocks 6.

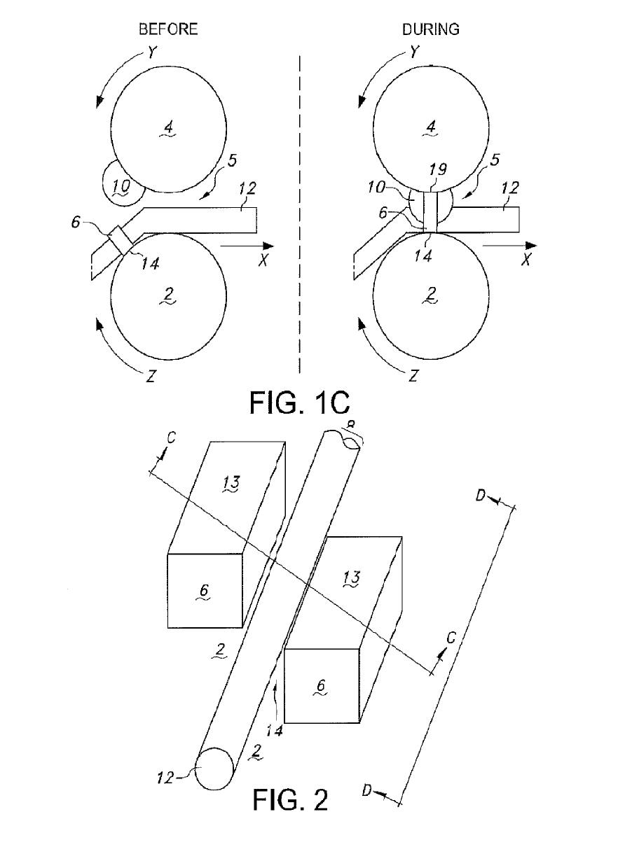

As best shown in FIG. 2, a set of two anvil blocks 6 are laid across the surface of the anvil wheel 2, forming the yarn space 8 and the yarn path 14. The anvil blocks 6 can be rectangular as shown, or the anvil blocks 6 may be any other desirable shape. For example, the anvil blocks 6 may be square, elliptical, oval or polygonal. Moreover, each face of the anvil blocks 6 may be of any shape. For example, in FIG. 2, the top face 13 of each anvil block is flat. However, each face may also have a texture or a shape. For example, the top face 13 may be rounded (FIG. 1B). The anvil blocks 6 in a set may be the same or different shapes from each other. The elastic yarn thread 12 lies between the anvil blocks 6, and the bottom of the elastic yarn thread 12 rests along the yarn path 14.

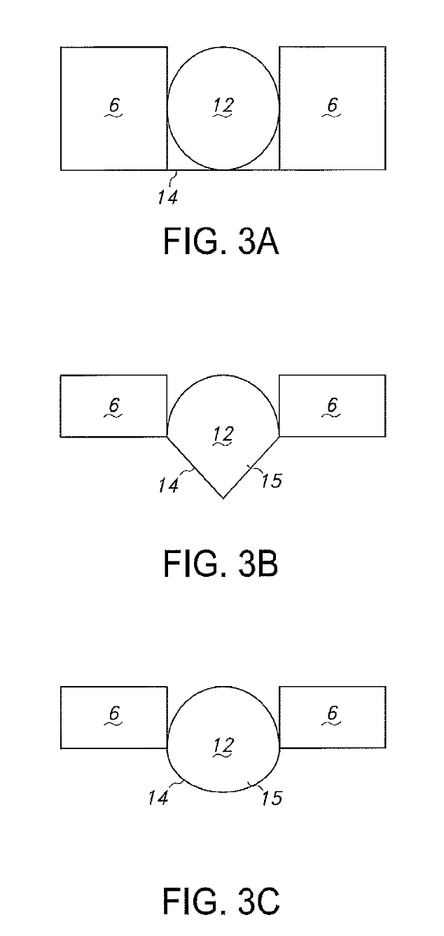

FIGS. 3A-C show a cross section of FIG. 2, along Line C-C. As best shown in FIG. 3A, the elastic yarn thread 12 fits snugly between the anvil blocks 6 and rests on the yarn path 14 which is approximately level with the surface of the anvil wheel (not shown). In an embodiment as shown in FIG. 3B, the elastic yarn thread 12 fits snugly between the anvil blocks 6 and rests snugly in an angular/profiled groove 15 cut into the yarn path 14. The height of the anvil blocks 6 may be reduced (as in, e.g., FIGS. 3B and 3C) to ensure a tight clamping of the elastic yarn thread 12. The depth of the groove 15 may also be increased or decreased to ensure a tight clamping of the elastic yarn thread 12. In another embodiment as shown in FIG. 3C, the elastic yarn thread 12 fits snugly between the anvil blocks 6 and rests snugly in a rounded groove 15 cut into the yarn path 14. The groove 15 in either embodiment serves to pinch the elastic yarn thread 12 more tightly between the anvil blocks 6 and to the yarn path 14 and anvil wheel 2, thereby reducing slips. When present, a convex member 10 (FIGS. 1B and 1C) on the horn wheel 4 (FIGS. 1B and 1C) presses the elastic yarn thread 12 down into the groove 15 and against the yarn path 14, thereby reducing slips.

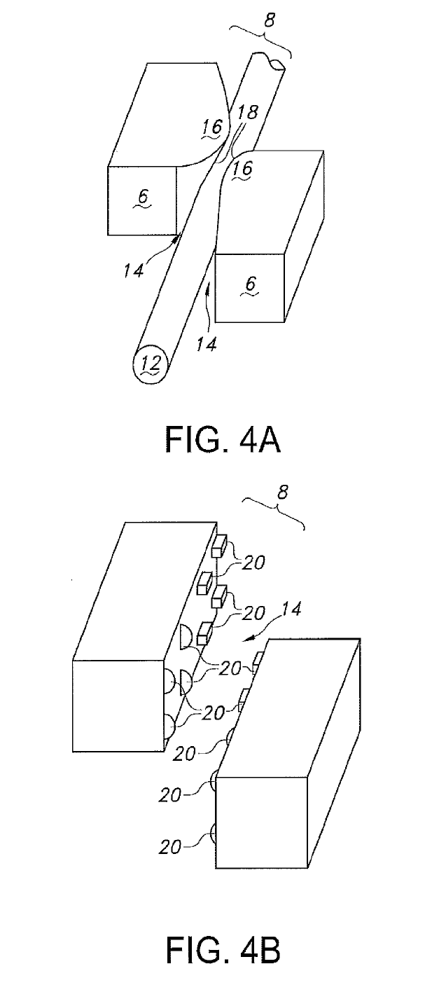

As best shown in FIG. 4A, the anvil blocks 6 may be modified anvil blocks that include one or more lateral protuberances 16 that extend into the yarn space 8 and across the yarn path 14, thereby pressing into the elastic yarn thread 12 at pinch points 18. The pinch points 18 assist in holding the elastic yarn thread 12 more tightly between the modified anvil blocks 6 and against the anvil wheel 2, thereby reducing slips. The lateral protuberances 16 need not be only rounded but may be of any useful shape that serves to create a pinch point 18. Additionally, the top of the lateral protuberance 16 and the bottom of the lateral protuberance 16 may equally extend into the yarn space 8 and across the yarn path 14. However, it is envisioned that the top of the lateral protuberance 16 may extend into the yarn space 8 and across the yarn path 14 either more or less than the bottom of the lateral protuberance 16, with the effect being that the side wall of the lateral protuberance 16 that faces the elastic yarn thread 12 may be slanted at an angle. Moreover, there may be one or more pinch points 18 created, depending on the shape of the lateral protuberance 16. Further, the shape of the lateral protuberance 16 on one modified anvil block 6 may be the same as or different from the shape of the lateral protuberance 16 on the other modified anvil block 6; in some embodiments, only one anvil block 6 is modified to include a lateral protuberance 16.

As best shown in FIG. 4B, the anvil blocks 6 may be modified to include one or more teeth 20 which protrude out from the side of the anvil blocks 6 into the yarn space 8 and across the yarn path 14. The teeth 20 may be rectangular or rounded; the teeth 20 may also be of any useful shape that will extend into the yarn space 8 and across the yarn path 14, thereby pressing into the side of the elastic yarn thread 12 (not shown) and creating pinch points 18 (not shown). Moreover, each modified anvil block 6 may have one or more teeth 20 of different shapes from each other and from the other modified anvil block 6. If two or more teeth 20 are present, they may be the same or different shapes. In some embodiments, only one anvil block 6 is modified to include one or more teeth 20, which may be the same or different shape from each other.

In some embodiments, one or both anvil blocks 6 may each be modified to have one or more lateral protuberances 16 and one or more teeth 20. In other embodiments, one anvil block 6 will be modified to have one or more lateral protuberances 16, and the other anvil block 6 will be modified to have one or more teeth 20. In another embodiment, one anvil block 6 will be modified to have one or more lateral protuberances 16 and/or one or more teeth 20, and the other anvil block 6 will have neither lateral protuberances nor teeth.

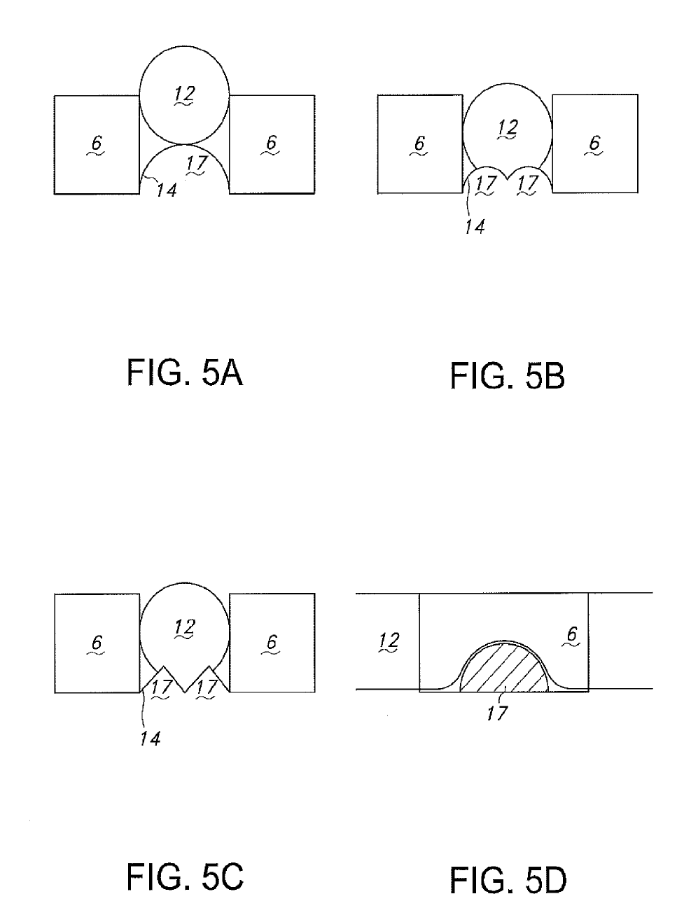

As best shown in FIG. 5A, the elastic yarn thread 12 fits snugly between the anvil blocks 6. In this embodiment, a convex circular ridge 17 is built up from the yarn path 14. Any other shape for the convex ridge known to be useful is also envisioned. In other embodiments, as shown, e.g., in FIGS. 5B and 5C, the convex ridge 17 may include multiple convexities which may be rounded (FIG. 5B), pointed/peaked (FIG. 5C) or any other convex shape known to be useful. In other embodiments, as shown, e.g., in FIG. 5D, the convex ridge 17 may be a convex lateral rounded ridge. In certain embodiments, as shown in, e.g., FIG. 5E, the convex ridge 17 may be perpendicular to the direction of the elastic yarn thread 18. In other embodiments, as shown in, e.g., FIG. 5F, the convex ridge 17 may be a convex tapered step ridge; the taller portion of the convex tapered step ridge 17 may be either toward or away from the direction of movement of the elastic yarn thread 12; the convex tapered step ridge 17 may also be positioned so that the taller portion is toward one anvil block 6 and the shorter portion is toward the other anvil block 6. It is also envisioned that any other shape known to be useful may be used. In practice, as best shown in the “Before” section of FIG. 5F, before the anvil wheel 2 and the horn wheel 4 come into contact with each other, the elastic yarn thread 12 may rest atop the convex tapered step ridge 17, and between the anvil blocks 6. As best shown in the “During” section of FIG. 5F, when the anvil wheel 2 and the horn wheel 4 come into contact with each other, the elastic yarn thread 12 is pressed against the convex tapered step ridge 17 and against the anvil wheel 2.

In some embodiments, the multiple convexities in the convex ridge 17 are different shapes, e.g., one rounded convexity and one pointed/peaked convexity. In some embodiments, there may be more than two convexities. When present, a convex member 10 (FIGS. 1B and 1C) on the horn wheel 4 (FIGS. 1B and 1C) presses the elastic yarn thread 12 down against the convex ridge 17 of the yarn path 14, thereby reducing slips. The heights of the convex ridge 17 and anvil blocks 6 may also be increased or decreased to ensure a tight clamping of the elastic yarn thread 12.

In certain embodiments, the yarn paths include structures similar or identical to the teeth or lateral protuberances that may be present on the modified anvil blocks; these structures may be instead of, or in addition to, any convex ridge that may be present.

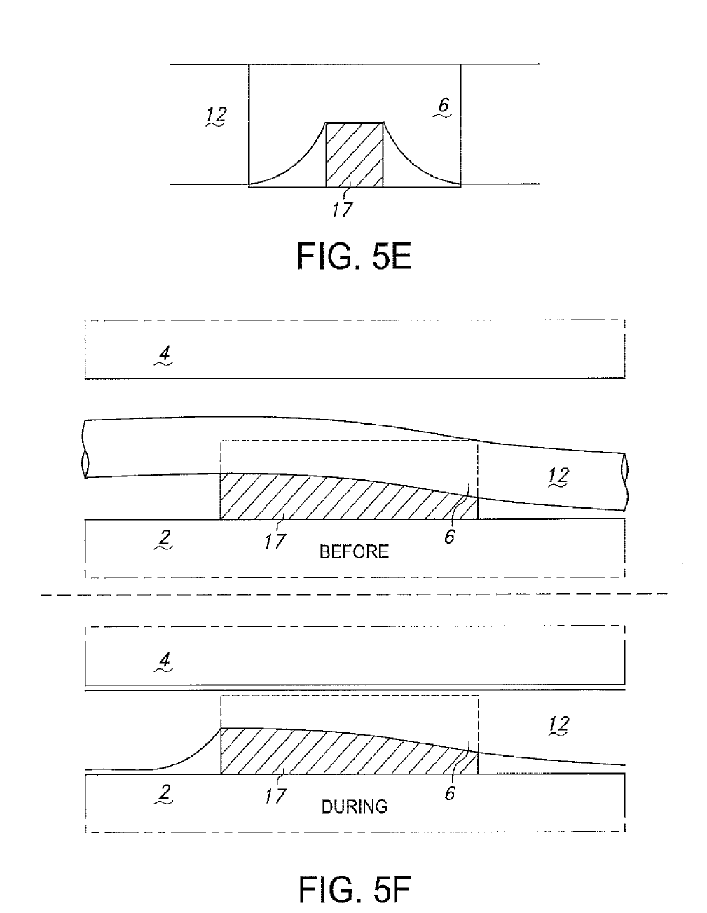

It should be noted that in actual practice, as best shown in FIG. 6, the elastic yarn thread 12 does not come into physical contact with any part of the anvil wheel 2 or its structures or horn wheel 4 or its structures because the elastic yarn thread 12 lies sandwiched between two textile sheets 22, 24. One textile sheet 24 is in contact with the anvil wheel 2 and its structures, and the other textile sheet 22 is in contact with the horn wheel 4 and its structures. Therefore, it is to be understood that all references to the elastic yarn thread 12 contacting either the anvil wheel 2 or its structures or horn wheel 4 or its structures implicitly means there is at least one layer of textile sheet 22, 24 between the elastic yarn thread 12 and wheel 2, 4. Similarly, any reference to the anvil wheel 2 or its structures contacting the horn wheel 4 or its structures is to be understood as implicitly meaning the contact is made through the textile sheets 22, 24.

During the production process, as best shown in FIG. 6, a first textile layer 22 and a second textile layer 24 are fed into the bonding machine 1 through the entry path 5; the anvil wheel 2 and horn wheel 4 are rotating in directions E and F, respectively. This causes the first and second textile layers 22, 24 to continuously move through the bonding machine 1, via the entry path 5. At least one elastic yarn thread 12 is simultaneously fed between the first and second textile layers 22, 24 and therefore through the anvil wheel 2 and horn wheel 4, via the entry path 5. The elastic yarn thread 12 is in a stretched state as it passes through the bonding machine 1, via the entry path 5. As the anvil wheel 2 and horn wheel 4 rotate, the first and second textile layers 22, 24 are brought into contact with each other in the entry path 5 at the point where the anvil blocks 6 and horn wheel 4 come into contact (FIGS. 1B and 1C) through the textiles, i.e., at the anvil/horn interface 19 (FIGS. 1B and 1C). If the bonding machine is a non-contact bonding machine, then the anvil/horn interface 19 (FIGS. 1B and 1C) is considered to be that point at which the anvil blocks 6 and the horn wheel 4 would have made contact (through the textiles) if positioned close enough to each other. At the anvil/horn interface 19 (FIGS. 1B and 1C), the two textiles 22, 24 are bonded together, thereby forming a bond point. The bond point serves to adhere the textiles 22, 24 to each other. The bond point also serves to squeeze the elastic yarn thread 12 in the finished product in order to reduce slippage. In an embodiment, the two textiles 22, 24 are bonded together via application of ultrasonic waves (which generate sufficient heat so as to cause the two textiles to adhere to each other). However, it is envisioned that any method of bonding known to those in the art may be utilized. The elastic band product 26 is therefore produced.

It should be noted that the elastic yarn thread 12 may encounter resistance to being placed into the yarn space 8 and along the yarn path 14, the resistance being created by the optional lateral protuberances 16 (FIG. 4A) or teeth 20 (FIG. 4B). This resistance may be overcome based on the tension supplied at the anvil contact point 30. That is, the tension on the elastic yarn thread 12 is sufficient to force the elastic yarn thread 12 past any lateral protuberances 16 (FIG. 4A) or teeth 20 (FIG. 4B), into the yarn space 8 and along the yarn path 14, and into the groove 15 (FIGS. 3B and 3C) or onto the convex ridge 17 (FIGS. 5A-5F) if present. Additional pressure may be supplied by the convex members 10 if present.

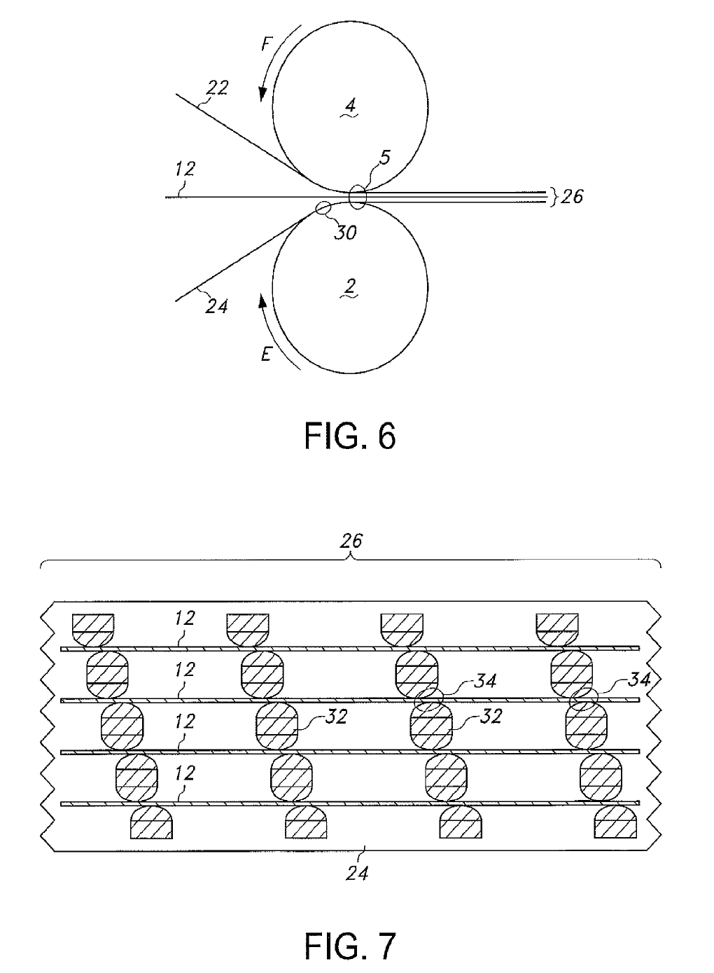

FIG. 7 illustrates an overhead view of an elastic band product 26 created according to the present invention. For convenience, the first textile layer has been removed to better illustrate the elastic yarn threads 12, although it is to be understood that the elastic yarn threads 12 would be hidden between the two textile layers 22, 24 in the finished product. In this embodiment, the anvil wheel (not shown) included modified anvil blocks (not shown) in sets of five. Therefore, the finished elastic band product 26 includes four separate elastic yarn threads 12 sandwiched between the first textile layer 22 (not shown) and the second textile layer 24. The elastic yarn threads 12 are held in position by pressure exerted onto the elastic yarn threads 12 by the bond points 32 at the pressure points 34, the pressure points 34 being the interface between the elastic yarn threads 12 and the bond points 32. The purpose of the pressure points 34 is to keep the elastic yarn thread 12 in place in the finished elastic band product 26 by reducing slippage. The shape of the bond points 32 may be dictated by the modification(s) to the anvil blocks 6 (not shown), i.e., the shaping and presence (or absence) of lateral protuberances 16 (not shown) and/or teeth 20 (not shown) used to produce the finished elastic band product 26.

It should be noted that in any embodiment of the present invention, the two textile sheets may be comprised of one textile sheet folded over both the elastic yarn thread(s) and itself. Moreover, the textile sheets may be of the same material as each other or may be of different materials. The textile sheets may be woven textile sheets or non-woven textile sheets. An examples of a non-woven textile sheet useful with the present invention is 15 GSM SMS polypropylene nonwoven material.

The elastic yarn threads may be comprised of any materials known to those of skill in the art, including polyurethane such as spandex or elastane, side-by-side polyester bicomponent fiber, elastomeric polyolefin, among others. If more than one elastic yarn thread is present, they may be the same material or of different materials.

The anvil and horn wheels, and their elements, may be comprised of any useful material. Examples of suitable metals include steel, titanium or titanium alloy, especially titanium alloy with homogenous density. The anvil and horn wheels (and their elements) may be comprised of the same materials as each other or of different materials.

The anvil and horn wheels may rotate at any useful speed, with suitable speeds being any speed that allows the wheels to match the linear speed of a production line, such as a diaper production line. In some embodiments, the wheels may be of different diameters; in such cases, those of skill in the art can easily calculate the required speeds for each wheel.

The ultrasonic waves used to bond the textile sheets together may be of any useful frequency and amplitude, applied for any useful amount of time. Suitable frequencies include about 20 to about 40 kHz, and the ultrasonic waves may be applied for about 0.1 millisecond to about 0.5 millisecond, or about 0.2 millisecond. The skilled artisan will be able to determine useful frequencies, durations and amplitudes that will enable adequate bonding while matching the linear speed of a production line, such as a diaper production line. If heat is used to bond the textile sheets together, the heat may be about ≤180° C., applied for <0.5 milliseconds. In some embodiments, the applied heat has a minimum temperature of about 120° C.

The angle or curve of the groove may be of any useful degree, such as 90 degrees.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

While certain and alternative embodiments of the present invention have been set forth for purposes of disclosing the invention, modifications to the disclosed embodiments may occur to those who are skilled in the art. Accordingly, the appended claims are intended to cover all embodiments of the invention and modifications thereof which do not depart from the spirit and scope of the invention.

All documents, patents and other literature referred to herein are incorporated by reference in their entirety.

The term “comprising” as may be used in the following claims is an open-ended transitional term that is intended to include additional elements not specifically recited in the claims. The term “consisting essentially of” as may be used in the following claims is a partially closed transitional phrase and is intended to include the recited elements plus any unspecified elements that do not materially affect the basic and novel characteristics of the claims. For example, the elastic band product may be embossed or printed with indicia and still be included in the meaning of “consisting essentially of”, even if not specifically recited. The term “consisting of” as may be used in the following claims is intended to indicate that the claims are restricted to the recited elements.

It should be noted that it is envisioned that any feature, element or limitation that is positively identified in this document may also be specifically excluded as a feature, element or limitation of an embodiment of the present invention.

1. A modified anvil block, wherein the modification is selected from the group consisting of an altered shape, inclusion of one or more teeth, inclusion of one or more lateral protuberances and combinations thereof.

2. An anvil wheel comprising at least one modified anvil block according to claim 1.

3. The anvil wheel of claim 2, further comprising at least one additional anvil block.

4. The anvil wheel of claim 3, wherein the at least one additional anvil block is modified, wherein the modification is selected from the group consisting of an altered shape, inclusion of one or more teeth, inclusion of one or more lateral protuberances and combinations thereof.

5. The anvil wheel of claim 3, further comprising at least one groove between the anvil blocks.

6. The anvil wheel of claim 5, wherein the groove is angular or rounded.

7. The anvil wheel of claim 3, further comprising at least one convex ridge between the anvil blocks.

8. The anvil wheel of claim 7, wherein the convex ridge is rounded, pointed/peaked or tapered.

9. A machine for producing elastic band products, comprising the anvil wheel of claim 2, and further comprising a horn wheel.

10. The machine of claim 9, wherein the horn wheel comprises a bonding means selected from the group consisting of a heat generating means and an ultrasonic wave generating means.

11. The machine of claim 9, wherein the horn wheel comprises at least one convex member.

12. The machine of claim 11, wherein the anvil wheel comprises at least one additional anvil block, and wherein the convex member is aligned between the anvil blocks.

13. The machine of claim 9, wherein the anvil wheel comprises at least one additional anvil block.

14. The machine of claim 9, wherein the machine is selected from the group consisting of a contact bonder and a non-contact bonder.

15. A method of producing an elastic band product comprising the steps of:
a) rotating the anvil wheel and the horn wheel of the machine according to claim 13,
b) feeding through an entry path of the machine at least two layers of textile sheets, wherein at least one elastic yarn thread is located between the at least two layers of textile sheets, parallel to the direction of feeding, and wherein the elastic yarn thread is positioned between the anvil blocks, and
c) bonding the at least two layers of textile sheets together, around the at least one elastic yarn thread, at an interface between the horn wheel and at least one of the anvil blocks.

16. The method of claim 15, wherein the elastic yarn thread is in a stretched state.

17. The method of claim 15, wherein the at least two layers of textile sheets are bonded by heat from a bonding means selected from the group consisting of a heat generating means and an ultrasonic wave generating means.

18. The method of claim 17, wherein the horn wheel comprises the bonding means.

19. An elastic band product produced by the method of claim 15.

20. A diaper comprising the elastic band product of claim 19, wherein the diaper is selected from the group consisting of disposable diapers and non-disposable diapers.

21. An apparatus for forming elastic bands comprising:
an anvil wheel and a horn wheel positioned to counter rotate so as to define an entry path therebetween;
the anvil wheel including at least one set of anvil blocks extending from a surface of the anvil wheel so as to accommodate a yarn in a yarn space defined therebetween; the yarn space having a dimension which is equal to or less than the diameter of the yarn as measured when the yarn is in a stretched state, so as to maintain the yarn in position in the yarn space while minimizing or reducing yarn slippage as the anvil wheel and the horn wheel counter rotate.

22. The apparatus of claim 21, wherein the horn wheel includes at least one convex member, wherein the convex member is aligned between the at least one set of anvil blocks.

23. The apparatus of claim 21, wherein at least one anvil block of the at least one set of anvil blocks includes a modification.

24. The apparatus of claim 23, wherein the modification is selected from the group consisting of an altered shape, inclusion of one or more teeth, inclusion of one or more lateral protuberances and combinations thereof.

25. The apparatus of claim 21, further comprising at least one groove between the at least one set of anvil blocks.

26. The apparatus of claim 25, wherein the groove is angular or rounded.

27. The apparatus of claim 21, further comprising at least one convex ridge between the at least one set of anvil blocks.

28. The apparatus of claim 27, wherein the convex ridge is rounded, pointed/peaked or tapered.

29. The apparatus of claim 21, wherein the horn wheel comprises a bonding means selected from the group consisting of a heat generating means and an ultrasonic wave generating means.
